(19) United States
(12) Patent Application Publication
(10) Pub. No.: US 2017/0283143 A1
(43) Pub. Date: Oct. 5, 2017

(54) BIODEGRADABLE VASE

(71) Applicant: N.Nicks Co., LLC, Longmont, CO (US)

(72) Inventors: Nora E. Nicks, Longmont, CO (US); Douglas A. Wigle, JR., Longmont, CO (US)

(73) Assignee: N.Nicks Co., LLC, Longmont, CO (US)

(21) Appl. No.: 15/474,922

(22) Filed: Mar. 30, 2017

Related U.S. Application Data
(60) Provisional application No. 62/315,379, filed on Mar. 30, 2016.

(51) Int. Cl. B65D 65/46; C07H 3/02; B65D 85/50; C07H 3/04

(52) U.S. Cl. CPC B65D 65/466; C07H 3/04; C07H 3/02; B65D 85/505

(57) ABSTRACT

A degradable container comprising a shaped body formed from a moldable composition including an amount of saccharide, an amount of saccharide-based solution, and an amount of vehicle.

### RELATED APPLICATIONS

This application claims priority to U.S. provisional patent application 62/315,379, entitled “Degradable Container”, filed on Mar. 30, 2016. The content of which is hereby incorporated by reference in its entirety.

### TECHNICAL FIELD

The disclosed embodiments generally relate to vases and more specifically to biodegradable vases that can be placed in the ground.

### BACKGROUND

It is common at cemeteries for people to leave objects, such as flowers, to show respect for loved ones. The objects are left leaning against headstones or laid on the ground. Some objects may be placed in conventional vases. These items may gather over time at locations in the cemetery or a worker may be required to gather the objects, sort the objects, and/or dispose/store the objects.

### I. SUMMARY

A broad object of a particular embodiment of the invention can be to provide a degradable container comprising a shaped body formed from a moldable composition including an amount of saccharide, an amount of saccharide-based solution, and an amount of vehicle.

Another broad object of a particular embodiment of the invention can be to provide a degradable container as described above, whereby the saccharide is selected from the group consisting of: monosaccharide, disaccharide, and oligosaccharide.

Another broad object of a particular embodiment of the invention can be to provide a method of making a degradable container, the method including forming a shaped body from a moldable composition including an amount of saccharide, an amount of saccharide-based solution, and an amount of vehicle.

Another broad object of a particular embodiment of the invention can be to provide a method of using a degradable container, the method including obtaining the degradable container comprising a shaped body formed from a moldable composition including: an amount of saccharide, an amount of saccharide-based solution, and an amount of vehicle; the shaped body having a shaped body internal surface which defines a shaped body interior cavity; disposing the degradable container proximate a ground surface; and disposing at least one containable element within the shaped body interior cavity.

Another broad object of a particular embodiment of the invention can be to provide a method of using a degradable container, the method further including allowing the degradable container to degrade into component parts.

Naturally, further objects of the invention are disclosed throughout other areas of the specification, drawings, and claims.

### II. A BRIEF DESCRIPTION OF THE DRAWINGS

FIG. 1A is an illustration of a method of using a particular embodiment of a degradable container for containing containable elements configured as a plurality of flowers and flower-supporting liquid.

FIG. 1B is an illustration of a method of using a particular embodiment of a degradable container for containing a containable element configured as a flag.

FIG. 2 is a perspective view of a particular embodiment of a degradable container.

FIG. 3 is a front view of a particular embodiment of a degradable container.

FIG. 4 is a rear view of a particular embodiment of a degradable container.

FIG. 5 is a first side view of a particular embodiment of a degradable container.

FIG. 6 is a second side view of a particular embodiment of a degradable container.

FIG. 7 is a first end view of a particular embodiment of a degradable container.

FIG. 8 is a second end view of a particular embodiment of a degradable container.

FIG. 9 is a schematic diagram of a method of making a particular embodiment of a degradable container.

### III. DETAILED DESCRIPTION OF PREFERRED EMBODIMENTS

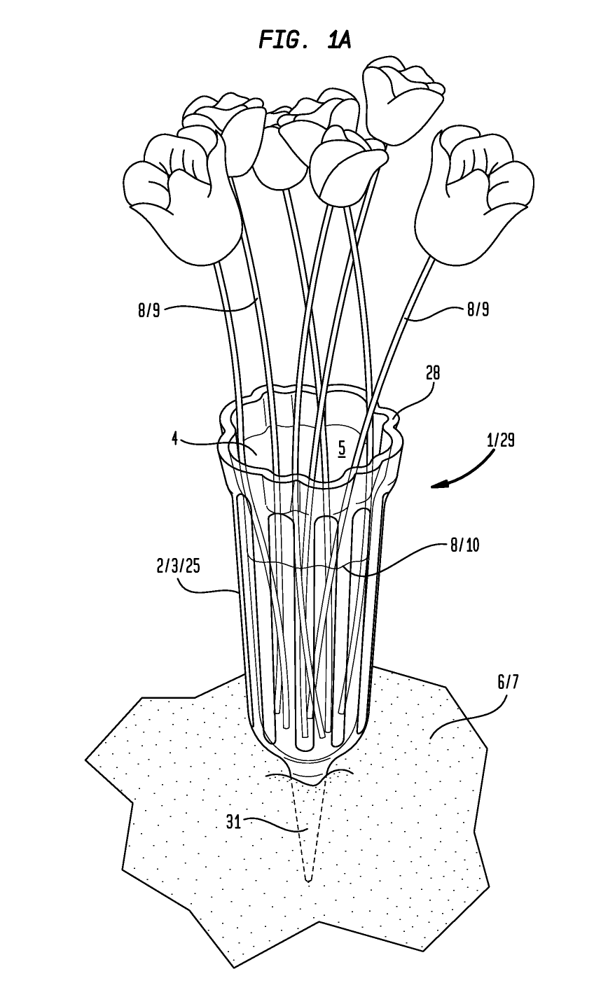

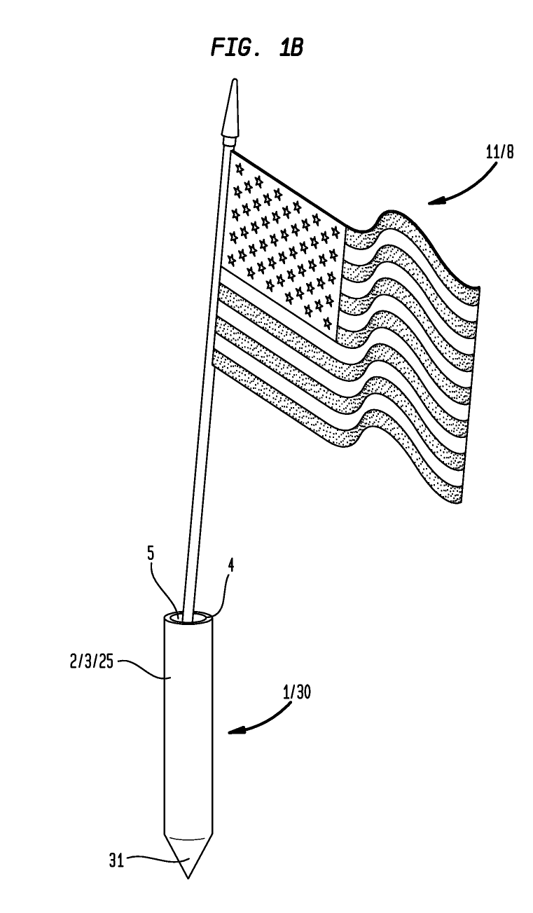

Now referring primarily to FIG. 1A and FIG. 1B, which illustrate methods of using a particular embodiment of a degradable container (1) having a shaped body (2) formed from a moldable composition (3), whereby the shaped body (2) has a shaped body internal surface (4) which defines a shaped body interior cavity (5). The degradable container (1) can be disposed proximate a ground surface (6), which can include soil (7), and one or more containable elements (8) can be disposed within the shaped body interior cavity (5). As non-limiting examples, containable elements (8) can include flowers (9) and flower-supporting liquids (10) such as water (as shown in the example of FIG. 1A) or a flag (11) (as shown in the example of FIG. 1B).

Following use (9), the degradable container (1) can degrade into component parts, whereby the component parts of the degradable container (1) can incorporate into the soil (7) and lessen the adverse effects of waste on the environment. As to particular embodiments, the component parts of the degradable container (1) can provide compost, which may be useful for fertilizing the soil (7).

As but one non-limiting example, the degradable container (1) may be useful in a cemetery, whereby after containing one or more containable elements (8), such as flowers (9) and flower-supporting liquids (10) or a flag (11), the degradable container (1) can degrade into component parts and incorporate into the soil (7) of the cemetery. As to particular embodiments, the degradable container (1) can be capable of being degraded, at least in part, by mechanical forces, such as forces exerted upon the degradable container (1) by a grass mower which mows grass within the cemetery.

The term “container” as used herein means any article, article of manufacture, receptacle, or vessel which can be used for containing one or more containable elements (8), such as objects (including, but not limited to, generally solid objects) or fluids (including, but not limited to, liquids). In addition to integrally-formed containers, containment products used in conjunction with containers are also intended to be included within the definition “container”. As but one illustrative example, a containment product which may be used in conjunction with a container can include a lid.

The term “moldable composition” as used herein means a composition capable of being molded into a shaped body (2) using any molding process known to one of ordinarily skill in the art.

The term “degradable” as used herein means capable of being degraded, decomposed, or broken down into component parts by the action of chemical or biological agents.

The term “biodegradable” as used herein means capable of being degraded, decomposed, or broken down by the action of biological agents, which can include microorganisms as a non-limiting example.

The term “compost” as used herein means a mixture of organic substances, which may be useful for fertilizing or improving soil.

Now referring primarily to Table 1, particular embodiments of the moldable composition (3) which form the shaped body (2) of the degradable container (1) can include formulations having the following raw materials: an amount of saccharide, an amount of saccharide-based solution, and an amount of vehicle. The raw materials can be admixed in the exemplary weight percentages (“Weight Percent”) shown in column two of Table 1. Numerous embodiments of the moldable composition (3) can be prepared by altering the weight percentages of the raw materials within the range weight percentages (“Range Weight Percent”) shown in column three of Table 1 with an amount of vehicle making up the balance.

TABLE 1

| Raw Material | Weight Percent | Range Weight Percent |
|---|---|---|
| Saccharide | 25% | 5-75 |
| Saccharide-based Solution | 25% | 5-75 |
| Vehicle | 50% | balance |

The saccharide can include a monosaccharide, such as ribose (CAS No: 50-69-1), xylose (CAS No: 58-86-6), fructose (CAS No: 57-48-7), glucose (dextrose) (CAS No: 50-99-7), galactose (CAS No: 59-23-4), mannose (CAS No: 31103-86-3), sorbose (CAS No: 87-79-6), or the like, or combinations thereof, all of which can be obtained from Sigma-Aldrich, 3050 Spruce Street, St. Louis, Mo. 63103, USA. As to other particular embodiments, the saccharide can include a disaccharide, such as sucrose (CAS No: 57-50-1), maltose (CAS No: 69-79-4), lactose (CAS No: 63-42-3), lactulose (CAS No: 4618-18-2), trehalose (CAS No: 99-20-7), cellobiose (CAS No: 528-50-7), or the like, or combinations thereof, all of which can be obtained from Sigma-Aldrich, 3050 Spruce Street, St. Louis, Mo. 63103, USA. As to yet other particular embodiments, the saccharide can include an oligosaccharide, which can be a saccharide polymer including a relatively lesser number of monosaccharides, whereby the number of monosaccharides is typically between three and nine.

As to particular embodiments, the saccharide can be selected from the group including or consisting of: monosaccharide, disaccharide, and oligosaccharide.

As to other particular embodiments, the saccharide can be selected from the group including or consisting of: monosaccharide and disaccharide.

As to other particular embodiments, the saccharide can be a disaccharide, such as sucrose.

The amount of saccharide can be generally in a range of between about 5% to about 75% by weight of the moldable composition (3); however, greater or lesser weight percents of the saccharide can be included in the moldable composition (3), depending upon the application.

As to particular embodiments, the amount of saccharide included in the moldable composition (3) can be selected from the group including or consisting of: between about 5% to about 15% by weight of the moldable composition (3); between about 10% to about 20% by weight of the moldable composition (3); between about 15% to about 25% by weight of the moldable composition (3); between about 20% to about 30% by weight of the moldable composition (3); between about 25% to about 35% by weight of the moldable composition (3); between about 30% to about 40% by weight of the moldable composition (3); between about 35% to about 45% by weight of the moldable composition (3); between about 40% to about 50% by weight of the moldable composition (3); between about 45% to about 55% by weight of the moldable composition (3); between about 50% to about 60% by weight of the moldable composition (3); between about 55% to about 65% by weight of the moldable composition (3); between about 60% to about 70% by weight of the moldable composition (3); and between about 65% to about 75% by weight of the moldable composition (3). As to the particular embodiment of the moldable composition (3) shown in Table 1, the amount of saccharide, for example sucrose, included in the moldable composition (3) can be about 25% by weight of the moldable composition (3).

The amount of saccharide included in the moldable composition (3) can be influenced by factors such as desired structural properties of the molded shaped body (2), desired physical appearance of the molded shaped body (2) (such as desired transparency, translucency, or opacity), desired rate of degradation of the degradable container (1), or the like, or combinations thereof.

The saccharide-based solution can include a saccharide-based syrup, as is generally known to one of ordinary skill in the art. As to particular embodiments, the saccharide-based syrup can be glucose syrup, which is formulated from hydrolyzed starch via the hydrolysis of starch. As to particular embodiments, the source of starch can be maize or corn to provide corn syrup (CAS No: 8029-43-7). As but one illustrative example, the corn syrup can be Karo® Light Corn Syrup, which is commercially available from ACH Food Companies, Inc., 717 Goodlett Farms Parkway, Memphis, Tenn. 38016, USA. As to other particular embodiments, the source of starch can be, as non-limiting examples, potatoes, wheat, barley, rice, cassava, or the like, or combinations thereof.

The amount of saccharide-based solution can be generally in a range of between about 5% to about 75% by weight of the moldable composition (3); however, greater or lesser weight percents of the saccharide-based solution can be included in the moldable composition (3), depending upon the application.

As to particular embodiments, the amount of saccharide-based solution included in the moldable composition (3) can be selected from the group including or consisting of: between about 5% to about 15% by weight of the moldable composition (3); between about 10% to about 20% by weight of the moldable composition (3); between about 15% to about 25% by weight of the moldable composition (3); between about 20% to about 30% by weight of the moldable composition (3); between about 25% to about 35% by weight of the moldable composition (3); between about 30% to about 40% by weight of the moldable composition (3); between about 35% to about 45% by weight of the moldable composition (3); between about 40% to about 50% by weight of the moldable composition (3); between about 45% to about 55% by weight of the moldable composition (3); between about 50% to about 60% by weight of the moldable composition (3); between about 55% to about 65% by weight of the moldable composition (3); between about 60% to about 70% by weight of the moldable composition (3); and between about 65% to about 75% by weight of the moldable composition (3). As to the particular embodiment of the moldable composition (3) shown in Table 1, the amount of saccharide-based solution, for example corn syrup, included in the moldable composition (3) can be about 25% by weight of the moldable composition (3).

The amount of saccharide-based solution included in the moldable composition (3) can be influenced by factors such as desired structural properties of the molded shaped body (2), desired physical appearance of the molded shaped body (2) (such as desired transparency, translucency, or opacity), desired rate of degradation of the degradable container (1), or the like, or combinations thereof.

The vehicle can include one or more of a numerous and wide variety of solvents in which the saccharide and saccharide-based solution can be solubilized or suspended.

As to particular embodiments, the vehicle can include water (CAS No: 7732-18-5), which can be filtered, de-ionized, distilled, or water otherwise filtered or purified.

The amount of vehicle can be an amount which forms the balance of the moldable composition (3). As to the particular embodiment of the moldable composition (3) shown in Table 1, the amount of vehicle, for example water, included in the moldable composition (3) can be about 50% by weight of the moldable composition (3).

The amount of vehicle included in the moldable composition (3) can be influenced by factors such as desired structural properties of the molded shaped body (2), desired physical appearance of the molded shaped body (2) (such as desired transparency, translucency, or opacity), desired rate of degradation of the degradable container (1), or the like, or combinations thereof.

Now referring primarily to Table 2, particular embodiments of the moldable composition (3) which form the shaped body (2) of the degradable container (1) can include formulations having the following raw materials: an amount of saccharide, an amount of saccharide-based solution, an amount of potassium bitartrate, and an amount of vehicle. The raw materials can be admixed in the exemplary weight percentages (“Weight Percent”) shown in column two of Table 2. Numerous embodiments of the moldable composition (3) can be prepared by altering the weight percentages of the raw materials within the range weight percentages (“Range Weight Percent”) shown in column three of Table 2 with an amount of vehicle making up the balance.

TABLE 2

| Raw Material | Weight Percent | Range Weight Percent |
|---|---|---|
| Saccharide | 25% | 5-75 |
| Saccharide-based Solution | 25% | 5-75 |
| Potassium bitartrate | 0.1% | 0.01-1% |
| Vehicle | 49.9% | balance |

The saccharide, saccharide-based solution, and vehicle can be as described above.

Potassium bitartrate (CAS No: 868-14-4), which is the potassium acid salt of tartaric acid, can be obtained from Sigma-Aldrich, 3050 Spruce Street, St. Louis, Mo. 63103, USA. As to particular embodiments, the potassium bitartrate may be formulated as cream of tartar, which can be obtained from McCormick & Co., Inc., 18 Loveton Circle, Sparks, Maryland 21152, USA.

The amount of potassium bitartrate can be generally in a range of between about 0.01% to about 1% by weight of the moldable composition (3); however, greater or lesser weight percents of potassium bitartrate can be included in the moldable composition (3), depending upon the application.

As to particular embodiments, the amount of potassium bitartrate included in the moldable composition (3) can be selected from the group including or consisting of: between about 0.01% to about 0.1% by weight of the moldable composition; between about 0.05% to about 0.15% by weight of the moldable composition; between about 0.1% to about 0.2% by weight of the moldable composition; between about 0.15% to about 0.25% by weight of the moldable composition; between about 0.2% to about 0.3% by weight of the moldable composition; between about 0.25% to about 0.35% by weight of the moldable composition; between about 0.3% to about 0.4% by weight of the moldable composition; between about 0.35% to about 0.45% by weight of the moldable composition; between about 0.4% to about 0.5% by weight of the moldable composition; between about 0.45% to about 0.55% by weight of the moldable composition; between about 0.5% to about 0.6% by weight of the moldable composition; between about 0.55% to about 0.65% by weight of the moldable composition; between about 0.6% to about 0.7% by weight of the moldable composition; between about 0.65% to about 0.75% by weight of the moldable composition; between about 0.7% to about 0.8% by weight of the moldable composition; between about 0.75% to about 0.85% by weight of the moldable composition; between about 0.8% to about 0.9% by weight of the moldable composition; between about 0.85% to about 0.95% by weight of the moldable composition; and between about 0.9% to about 1% by weight of the moldable composition. As to the particular embodiment of the moldable composition (3) shown in Table 2, the amount of potassium bitartrate included in the moldable composition (3) can be about 0.1% by weight of the moldable composition (3).

The amount of potassium bitartrate included in the moldable composition (3) can be influenced by factors such as desired structural properties of the molded shaped body (2), desired physical appearance of the molded shaped body (2) (such as desired transparency, translucency, or opacity), desired rate of degradation of the degradable container (1), or the like, or combinations thereof.

As to particular embodiments, the moldable composition (3) can, but need not necessarily, further include an amount of one or more binding agents, which may aid in rigidity and decrease spoilage (due to microorganisms). As to particular embodiments, the binding agent can include a salt, such as an alkali metal salt or an alkaline earth metal salt. As an illustrative example, the binding agent can be sodium chloride, such as Diamond Crystal® Salt, which is commercially available from Cargill®, P.O. Box 5621, Minneapolis, Minn. 55440, USA.

The binding agent can be generally included in an amount of about 0.1% to about 5% by weight of the moldable composition (3); however, greater or lesser weight percents of the binding agent can be included in the moldable composition (3), depending upon the application.

As to particular embodiments, the moldable composition (3) can, but need not necessarily, further include an amount of one or more mold release agents, which may facilitate release or detachment of the shaped body (2) from a mold during the molding process.

As to particular embodiments, the mold release agent can include animal fats, vegetable fats, lecithin, or the like, or combinations thereof. As an illustrative example, the mold release agent can include shortening, such as Crisco® Shortening, which is commercially available from The J.M. Smucker Company, 1 Strawberry Lane, Orrville, Ohio 44667, USA. As another illustrative example, the mold release agent can include gluten. As yet another illustrative example, the mold release agent can include a combination of shortening and gluten.

As to other particular embodiments, the mold release agent can include glycerol, microcrystalline wax, fatty alcohols, ethylene glycol, propylene glycol, glycerin, 1,3-propanediol, 1,2-butandiol, 1,3-butandiol, 1,4-butanediol, 1,5-pentandiol, 1,5-bexandiol, 1,6-hexandiol, 1,2,6-hexantriol, 1,3,5-hexantriol, neopentylglycol, sorbitol acetate, sorbitol diacetate, sorbitol monoethoxylate, sorbitol diethoxylate, sorbitol hexaethoxylate, sorbitol dipropoxylate, arrunosorbitol, trihydroxymethylaminomethane, glucose/PEG, the reaction product of ethylene oxidewith glucose, trimethylolpropane monoethoxylate, mannitol monoacetate, mannitol monoethoxylate, butyl glucoside, glucose monoethoxylate, a-methyl glucoside, the sodium salt of carboxymethylsorbitol, polyglycerol monoethoxylate, erythritol, pentaerythritoi, arabitol, adonitol, xylitol, mannitol, iditol, galactitl, ailitol, sorbitol, polyhydric alcohols generally, esters of glycerin, formamide, N-methylformamide, DMSO, mono- and diglycerides, alkylarruides, polyols, trimethylolpropane, polyvinylalcohol with from 3 to 20 repeating units, polyglycerols with from 2 to 10 repeating units, and derivatives of the foregoing. Examples of derivatives include ethers, thiethers, inorganic and organic esters, acetals, oxidation products, amides, and amines.

The mold release agent can be generally included in an amount of about 1% to about 10% by weight of the moldable composition (3); however, greater or lesser weight percents of the mold release agent can be included in the moldable composition (3), depending upon the application.

As to particular embodiments, the moldable composition (3) can, but need not necessarily, further include an amount of one or more auxiliary agents, such as preservatives, coloring agents, fragrances, flavors, or the like.

As non-limiting examples, preservatives can include ethanol, citric acid, ascorbic acid, sodium benzoate, raisin paste, or the like, or combinations thereof.

As non-limiting examples, coloring agents can include natural coloring agents, such as saffron, paprika, beetroot, crocein, carotene, or the like, or combinations thereof.

As to particular embodiments, the moldable composition (3) can, but need not necessarily, further include an amount of wood fiber, wood flour, products made from wood (such as paper products), or the like, or combinations thereof.

A method of making a degradable container (1) can include forming a shaped body (2) from a moldable composition (3) comprising the following raw materials: an amount of saccharide, an amount of saccharide-based solution, and an amount of vehicle. As to particular embodiments, the moldable composition (3) can, but need not necessarily, further include one or more of the additional components described above, depending upon the application.

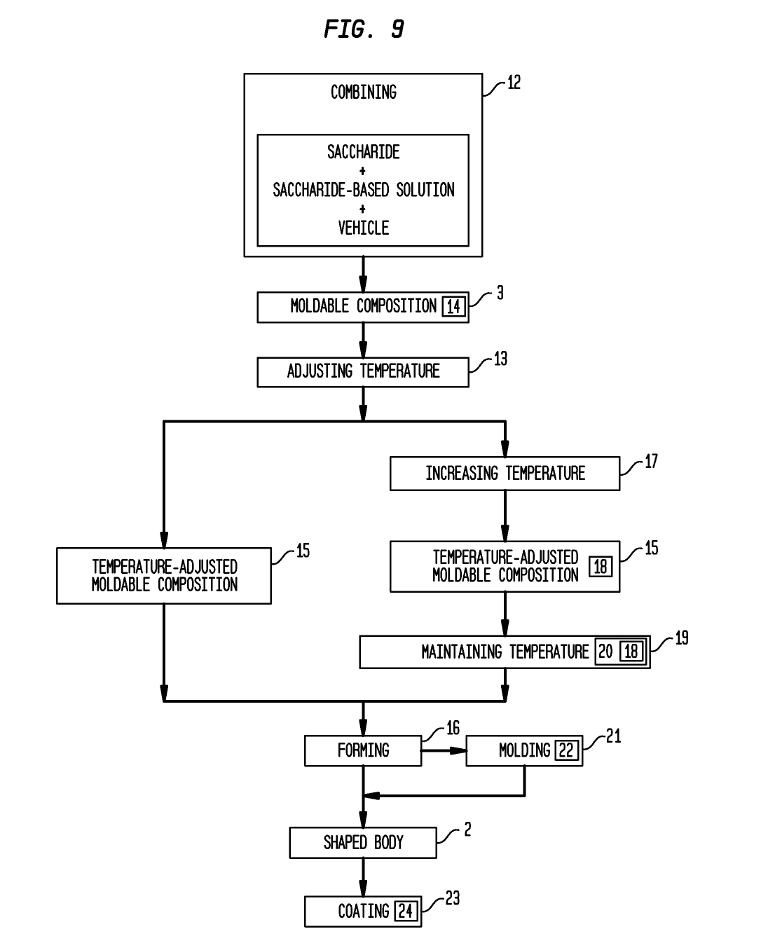

Now referring primarily to FIG. 9, as to particular embodiments, forming the shaped body (2) from the moldable composition (3) can include combining (12) the amount of saccharide, the amount of saccharide-based solution, and the amount of vehicle to provide the moldable composition (3); adjusting (13) a temperature (14) of the moldable composition (3) to provide a temperature-adjusted moldable composition (15); and forming (16) the temperature-adjusted moldable composition (15) into the shaped body (2).

As used herein, the term “combining or combination” refers to any method of putting two or more materials together. Such methods include, but are not limited to, mixing, blending, commingling, concocting, homogenizing, ultrasonic homogenizing, incorporating, intermingling, fusing, joining, shuffling, stirring, coalescing, integrating, confounding, uniting, creating a solution of two or more materials, creating a suspension of two immiscible materials, such as an emulsion, via any number of means, or the like, or combinations thereof.

Again referring primarily to FIG. 9, as to particular embodiments, adjusting (13) the temperature (14) of the moldable composition (3) to provide the temperature-adjusted moldable composition (15) can, but need not necessarily, include increasing (17) the temperature (14) of the moldable composition (3) to an increased temperature (18).

As to particular embodiments, the increased temperature (18) can be in a range of between about 50° Celsius to about 250° Celsius (about 122° Fahrenheit to about 482° Fahrenheit). As but one illustrative example, the increased temperature can be about 149° Celsius (about 300° Fahrenheit).

Again referring primarily to FIG. 9, as to particular embodiments, the method of making the degradable container (1) can, but need not necessarily, further include maintaining (19) the temperature-adjusted moldable composition (15) at the increased temperature (18) for a first time period (20) while the temperature-adjusted moldable composition (15) is under conditions of mixing, which may facilitate further combining.

As to particular embodiments, the first time period (20) can be in a range of between about 5 minutes to about 120 minutes. As but one illustrative example, the first time period (20) can be about in a range of between about 20 minutes to about 25 minutes.

Again referring primarily to FIG. 9, as to particular embodiments, forming the temperature-adjusted moldable composition (15) into the shaped body (2) can, but need not necessarily, include molding (21) the temperature-adjusted moldable composition (15) into the shaped body (2), whereby molding (21) can include any molding process known to one of ordinarily skill in the art. As non-limiting examples, molding processes can include: casting, extruding, press molding, injection molding, printing, additive printing, or the like, or combinations thereof. The viscosity and other forming characteristics of the moldable composition (3) may be tailored to the particular forming or molding process to be employed in accordance with techniques known to one of ordinary skill in the art.

The shaped body (2) can be molded as one piece or assembled from a plurality of pieces into an embodiment of the shaped body (2). As but one illustrative example, the shaped body (2) can be formed as a one-piece shaped body (2) from casting or injection molding by respectively casting or injecting the moldable composition into a mold (22) having a mold cavity corresponding to the one-piece shaped body (2).

Again referring primarily to FIG. 9, as to particular embodiments, the method of making the degradable container (1) can, but need not necessarily, further include coating (23) one or more surfaces of the shaped body (2) with one or more coating agents (24).

During or after any of the forming (16) or molding (21) processes, one or more degradable coating agents (24) can be applied to or incorporated within one or more surfaces of the shaped body (2) for any desired purpose, including as non-limiting examples: to make one or more surfaces of the shaped body (2) substantially water resistant or waterproof to impart a glossiness to one or more surfaces of the shaped body (2); to make the shaped body (2) more flexible; or the like; or combinations thereof. As to particular embodiments, coating agents (24) can be used to alter the surface characteristics of the shaped body (2), for example by sealing the shaped body (2) to provide protection against moisture, acids, bases, oils, organic solvents, polar solvents, or the like, or combinations thereof.

As to particular embodiments, a portion or an entirety of a shaped body external surface (25), a portion or an entirety of the shaped body internal surface (4), or combinations thereof, can be coated with one or more coating agents (24).

As to particular embodiments, the coating agent (24) can include cellulose, gums such as guar or Arabic, waxes, starches, gluten, egg albumin, egg white, shellac, whey protein, lecithin, or the like, or combinations thereof.

Water resistance can be achieved via application of a water resistant coating agent (24) to one or more surfaces of the shaped body (2). As non-limiting examples, water resistant coating agents (24) can include: zein (a biodegradable material isolated from corn); polylactic acid (PLA, polymers of lactic acid from fermentation feed stock); polyhydroxyalkanoates (PHA, linear polyesters from microbial fermentation); bacterial cellulose; chitosan-based polymers (for example, from shell fish wastes); waxes; wax-based coatings; oils; oil-based coating; or the like; or combinations thereof.

The one or more coating agents (24) can be applied to or incorporated within one or more surfaces of the shaped body (2) by any coating process known to one of ordinary skill in the art, including as non-limiting examples: spraying; dipping; or the like; or combinations thereof. Alternatively, one or more coating agents (24) may be cast or otherwise formed into a film or layer, which may then be applied to the shaped body (2).

As to particular embodiments of the degradable container (1), it may be desirable to apply print or other indicia, such as trademarks, product information, container specifications, or logos, on one or more surfaces of the shaped body (2). This can be accomplished using any conventional printing means or processes known in the art of printing products, including planographic, relief, intaglio, porous, impactless printing, or the like, or combinations thereof. Conventional printers include offset, Van Dam, laser, direct transfer contact, and therrographic printers. However, essentially any manual or mechanical means can be used.

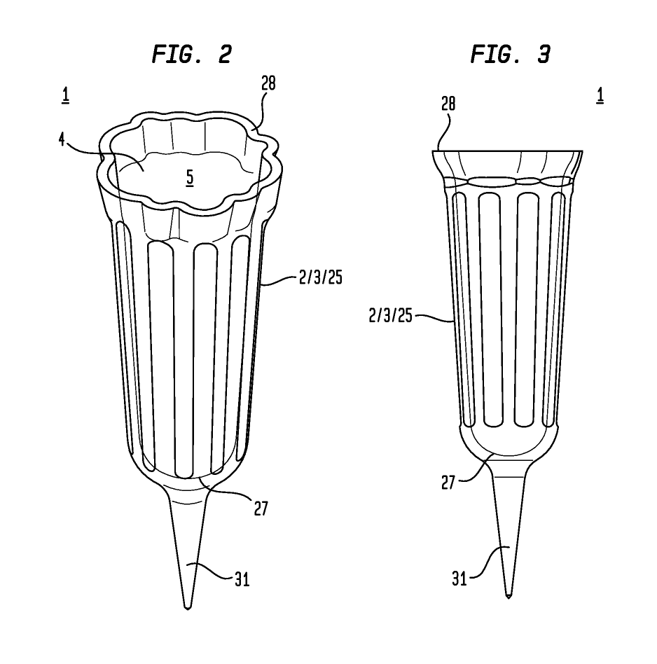

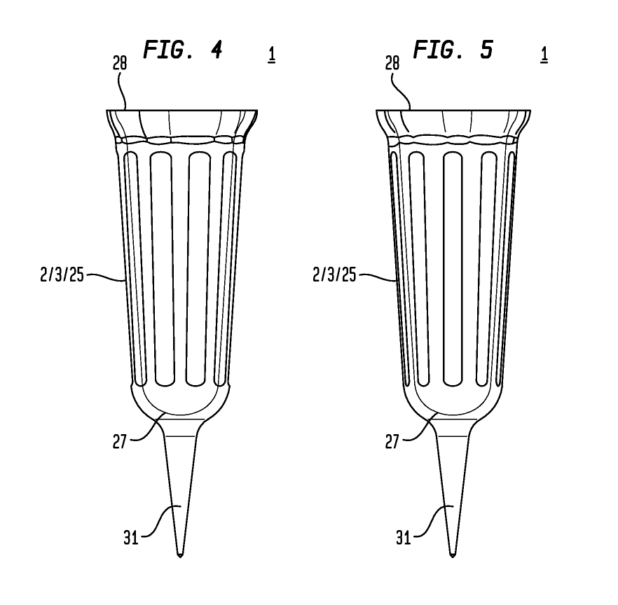

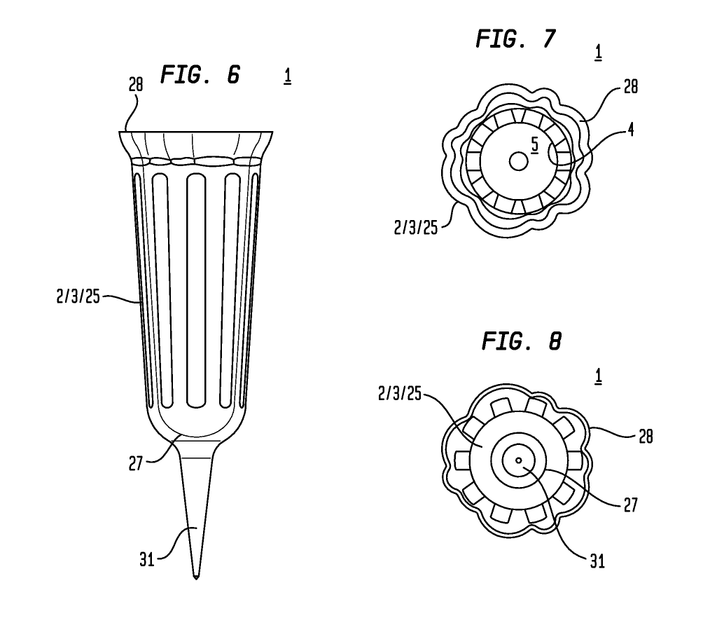

Now referring primarily to FIG. 2 through FIG. 8, as to particular embodiments, the degradable container (1) can include a shaped body (2) having opposing shaped body external and internal surfaces (25)(4), whereby the shaped body internal surface (4) defines a shaped body interior cavity (5). The shaped body (2) can further have a shaped body closed end (27) opposite a shaped body open end (28), whereby the shaped body open end (28) communicates with the shaped body interior cavity (5).

The shaped body (2) can have any of a numerous and wide variety of configurations, depending upon the application. As a non-limiting example, the shaped body (2) can be configured as a vase (29), whereby the shaped body (2) can be an elongated shaped body (2) which has a generally circular cross-section and inwardly tapers toward the shaped body closed end (27) (as shown in the examples of FIG. 1A and FIG. 2 through FIG. 8). As another non-limiting example, the shaped body (2) can be configured as a flag holder (30), whereby the shaped body (2) can be an elongated shaped body (2) which has a generally circular cross-section and inwardly tapers toward the shaped body closed end (27) (as shown in the example of FIG. 1B).

As to particular embodiments, the degradable container (1) can further include a stake element (31) coupled to the shaped body (2) proximate the shaped body closed end (27), whereby the stake element (31) can facilitate staking of the shaped body (2) within a ground surface (6). As to particular embodiments, the stake element (31) can be formed from the moldable composition (3) as described above for the shaped body (2).

As to particular embodiments, the shaped body (2) and the stake element (31) can be molded as a one-piece construct from casting or injection molding by respectively casting or injecting the moldable composition (3) into a mold (22) having a mold cavity corresponding to the one-piece construct including the shaped body (2) and the stake element (31).

A method of using a degradable container (1) can include obtaining the degradable container (1), configured as described above, whereby the shaped body (2) has a shaped body internal surface (4) which defines a shaped body interior cavity (5); disposing the degradable container (1) proximate a ground surface (6), which can include soil (7); and disposing one or more containable elements (8), for example flowers (9) and flower-supporting liquids (10) such as water (as shown in the example of FIG. 1A) or a flag (11) (as shown in the example of FIG. 1B), within the shaped body interior cavity (5).

As to particular embodiments, the method of using the degradable container (1) can, but need not necessarily, further include allowing the degradable container (1) to degrade into component parts (for example, following expiration of the flowers (9) contained within the shaped body interior cavity (5)).

As to particular embodiments, the degradable container (1) can degrade into component parts in a second time period selected from the group including or consisting of: less than two years; less than one year; less than ten months; less than eight months; less than six months; less than four months; less than three months; less than two months; less than one month; less than three weeks; less than two weeks; and less than one week.

Following degradation, the component parts of the degradable container (1) can, but need not necessarily, incorporate into the soil (7) and lessen the adverse effects of waste on the environment. As to particular embodiments, the components of the degradable container (1) can provide compost, which may be useful for fertilizing the soil (7).

As can be easily understood from the foregoing, the basic concepts of the present invention may be embodied in a variety of ways. The invention involves numerous and varied embodiments of a degradable container and methods for making and using such degradable containers, including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “mold” should be understood to encompass disclosure of the act of “molding”—whether explicitly discussed or not—and, conversely, were there effectively disclosure of the act of “molding”, such a disclosure should be understood to encompass disclosure of a “mold” and even a “means for molding”. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the degradable containers herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A degradable container comprising:
a shaped body formed from a moldable composition including:
an amount of saccharide;
an amount of saccharide-based solution; and
an amount of vehicle.

2. The degradable container of claim 1, wherein said saccharide is selected from the group consisting of: monosaccharide, disaccharide, and oligosaccharide.

3. A method of making a degradable container, said method comprising:
forming a shaped body from a moldable composition including:
an amount of saccharide;
an amount of saccharide-based solution; and
an amount of vehicle.

4. A method of using a degradable container, said method comprising:
obtaining said degradable container comprising:
a shaped body formed from a moldable composition including:
an amount of saccharide;
an amount of saccharide-based solution; and
an amount of vehicle;
said shaped body having a shaped body internal surface which defines a shaped body interior cavity;
disposing said degradable container proximate a ground surface; and
disposing at least one containable element within said shaped body interior cavity.

5. The method of claim 4, further comprising allowing said degradable container to degrade into component parts.
